(19) United States
(12) Patent Application Publication
(10) Pub. No.: US 2026/0026962 A1
(43) Pub. Date: Jan. 29, 2026

(54) URINARY ASSISTANCE DEVICE FOR USERS WITH FEMALE URETHRAL ANATOMY

(71) Applicant: ARIZONA BOARD OF REGENTS ON BEHALF OF THE UNIVERSITY OF ARIZONA, Tucson, AZ (US)

(72) Inventor: Trent Tresch, Oracle, AZ (US)

(21) Appl. No.: 19/276,410

(22) Filed: Jul. 22, 2025

Related U.S. Application Data
(60) Provisional application No. 63/674,453, filed on Jul. 23, 2024.

(51) Int. Cl. A61F 5/455; A61F 5/44

(52) U.S. Cl. CPC A61F 5/4556; A61F 5/4401; A61F 5/4405; A61F 5/4408

(57) ABSTRACT

An external urinary assistance device for permitting urination of a user having female urethral anatomy. The device includes a garment configured to fit around a pelvis of the user. The device further includes a primary reservoir operatively coupled to the garment, configured to fluidly couple to a urethra of the user. The device further includes a flexible lip disposed around a border of the primary reservoir, configured to create a waterproof seal between the user and the primary reservoir. The device further includes a plurality of tubes fluidly coupled to the primary reservoir configured to siphon fluid from the primary reservoir.

### CROSS-REFERENCE TO RELATED APPLICATIONS

This application is a non-provisional and claims benefit of U.S. Provisional Application No. 63/674,453 filed Jul. 23, 2024, the specification(s) of which is/are incorporated herein in their entirety by reference.

### FIELD OF THE INVENTION

The present invention is directed to an external urinary assistance device for allowing users with female urethral anatomy to urinate while standing, sitting, walking, or any other activity and in any body position. For example, the unit could be used, but is not limited to use, by someone while flying aircraft, flying spacecraft, used inside of a pressure suit or spacesuit, by race car drivers, climbing mountains, or used in extreme conditions like those found in polar locations or undersea in a submarine.

### BACKGROUND OF THE INVENTION

People sometimes spend extended lengths of time in conditions that disallow the ability to urinate. It is important, especially in extreme circumstances such as military and space flight, to be adequately able to void the bladder while performing tasks that could impact human safety. The ability to void at any time also negates medical issues such as urinary tract infections, ischuria, and the weakening of the bladder muscles.

Staying hydrated has many benefits such as helping the body maintain a normal temperature, lubricate joints, protect sensitive tissues, and even help prevent decompression and decompression sickness. With adequate hydration, however, people often need to urinate, sometimes while in limited circumstances. Males and those with anatomically similar urethral capabilities to that of the male gender can use external catheters in these circumstances, while those with anatomically similar urethral capabilities to that of the female gender traditionally either had to rely on less desirable alternatives (e.g., using diapers or internal catheters, artificially limiting urine output by restricting hydration or increasing sodium intake), or wait until they have the capacity to void. As mentioned, this can cause health and safety problems to the individual and others who may be relying on them.

### BRIEF SUMMARY OF THE INVENTION

It is an objective of the present invention to provide devices and systems that allow for external urinary assistance devices for allowing users with female urethral anatomy to urinate while standing, sitting, walking, or any other activity and in any body position, as specified in the independent claims. Embodiments of the invention are given in the dependent claims. Embodiments of the present invention can be freely combined with each other if they are not mutually exclusive.

In some embodiments, the present invention features an external urinary assistance device for permitting urination of a user having female urethral anatomy. In some embodiments, the device may comprise a garment configured to fit around a pelvis of the user. The device may further comprise a primary reservoir operatively coupled to the garment, configured to fluidly couple to a urethra of the user. The device may further comprise a flexible lip disposed around a border of the primary reservoir, configured to create a waterproof seal between the user and the primary reservoir. The device may further comprise a plurality of tubes fluidly coupled to the primary reservoir configured to siphon fluid from the primary reservoir.

One of the unique and inventive technical features of the present invention is the implementation of a wearable garment comprising a flexible inlet capable of forming a waterproof seal to the urethra of a user. Without wishing to limit the invention to any theory or mechanism, it is believed that the technical feature of the present invention advantageously provides for users with female urethral anatomy to freely urinate in any body position. None of the presently known prior references or work has the unique inventive technical feature of the present invention.

Any feature or combination of features described herein are included within the scope of the present invention provided that the features included in any such combination are not mutually inconsistent as will be apparent from the context, this specification, and the knowledge of one of ordinary skill in the art. Additional advantages and aspects of the present invention are apparent in the following detailed description and claims.

### BRIEF DESCRIPTION OF THE SEVERAL VIEWS OF THE DRAWING(S)

The features and advantages of the present invention will become apparent from a consideration of the following detailed description presented in connection with the accompanying drawings in which:

FIG. 1A shows a schematic diagram of a first embodiment of the urinary assistance device of the present invention.

FIG. 1B shows a schematic diagram of a second embodiment of the urinary assistance device of the present invention.

FIG. 2 shows a schematic diagram of the urinary assistance device of the present invention in use.

FIG. 3 shows a side view of the urinary assistance device while in use.

### DETAILED DESCRIPTION OF THE INVENTION

Following is a list of elements corresponding to a particular element referred to herein:
100 device
110 garment
120 primary reservoir
125 flexible lip
130 tubes
132 siphon tube
134 equalizing tube
135 one-way valve
140 secondary reservoir
150 fitting

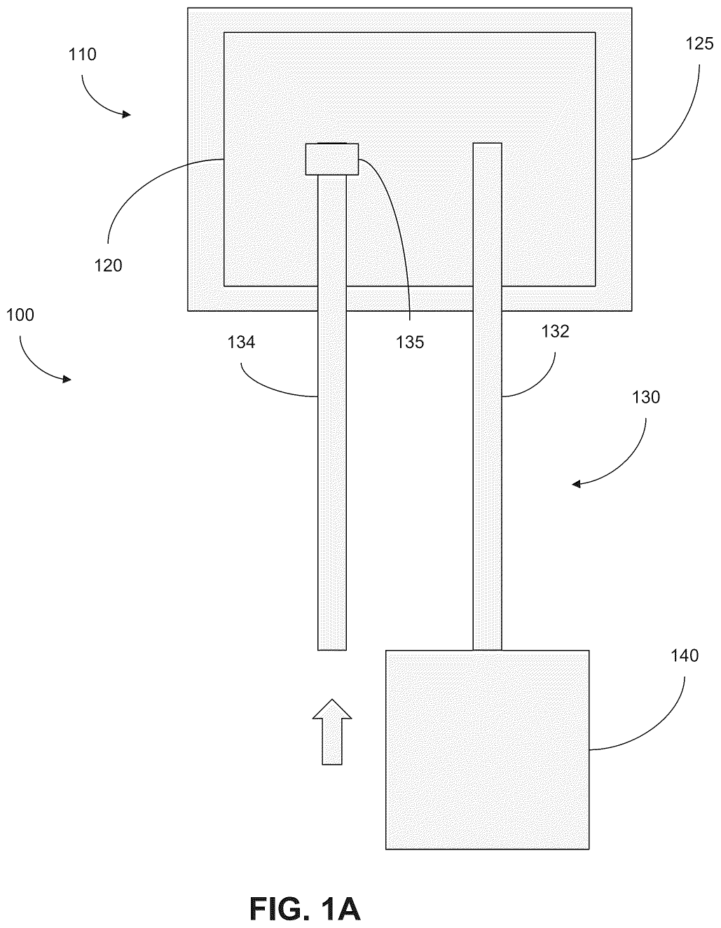

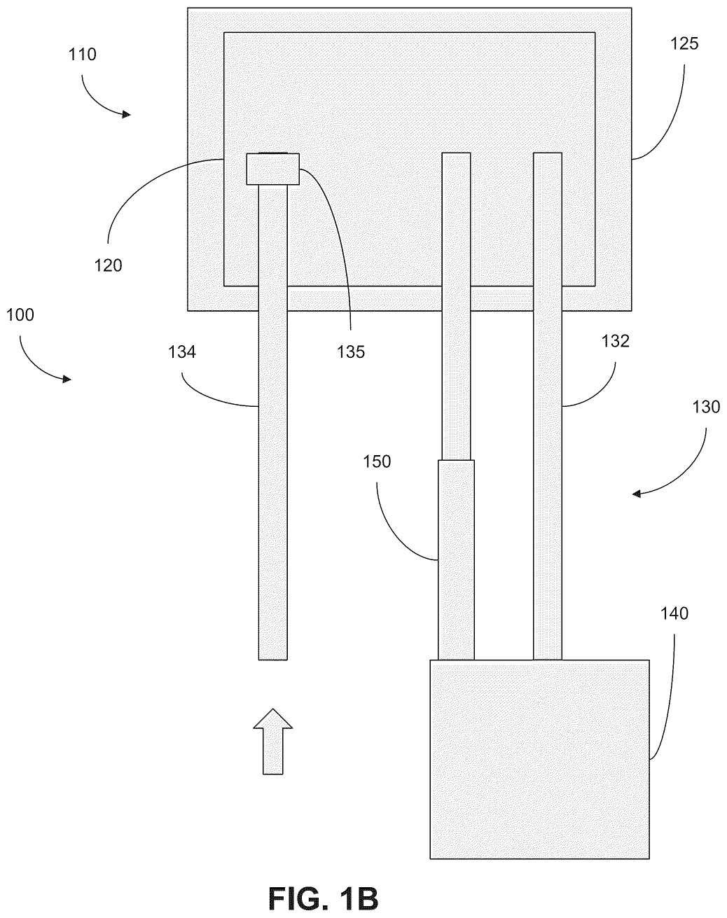

Referring now to FIGS. 1A-1B, the present invention features an external urinary assistance device (100) for permitting urination of a user having female urethral anatomy. In some embodiments, the device (100) may comprise a garment (110) configured to fit around a pelvis of the user. The device (100) may further comprise a primary reservoir (120) operatively coupled to the garment (110), configured to fluidly couple to a urethra of the user. The device (100) may further comprise a flexible lip (125) disposed around a border of the primary reservoir (120), configured to create a waterproof seal between the user and the primary reservoir (120). The device (100) may further comprise a plurality of tubes (130) fluidly coupled to the primary reservoir (120) configured to siphon fluid from the primary reservoir (120).

In some embodiments, each tube of the plurality of tubes may comprise a proximal end and a distal end. In some embodiments, each tube of the plurality of tubes may be fluidly coupled to the primary reservoir (120) at the proximal end. In some embodiments, the device (100) may further comprise a secondary reservoir (140). The distal end of at least one tube (132) of the plurality of tubes (130) may be fluidly coupled to the secondary reservoir (140) such that the fluid is directed from the primary reservoir (120) to the secondary reservoir (140). In some embodiments, at least one tube (134) of the plurality of tubes (130) may comprise a one-way valve (135) configured to direct an equalizing substance into the primary reservoir (120) such that an internal pressure of the primary reservoir (120) is balanced. In some embodiments, the equalizing substance may comprise air, water, oxygen gas, or a combination thereof.

In some embodiments, the flexible lip (125) may comprise an adhering component configured to adhere the primary reservoir (120) to the user such that the waterproof seal is created. The adhering component may comprise glue, tape, or a combination thereof. In some embodiments, the garment (110) may comprise an absorbent material (e.g. super-absorbent polymers). In some embodiments, the flexible lip (125) may comprise silicon, natural rubber, engineered rubber, or a combination thereof. In some embodiments, the device (100) may be configured to allow the user to urinate while sitting or standing in a vehicle, the vehicle comprising an aircraft, a spacecraft, an automobile, or a train. In some embodiments, at least one tube of the plurality of tubes (130) may comprise a fitting (150) disposed at an end of the tube. In some embodiments, the fitting (150) may comprise an extension tube, a one-way valve, a push-to-connect fitting compatible with an extension tube, a quick disconnect fitting compatible with an extension tube, or a combination thereof.

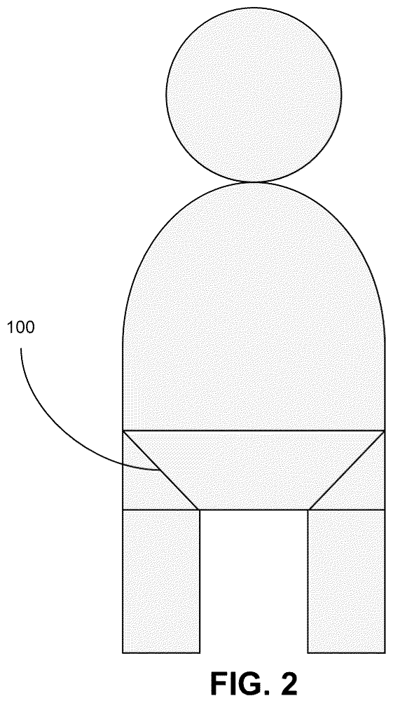

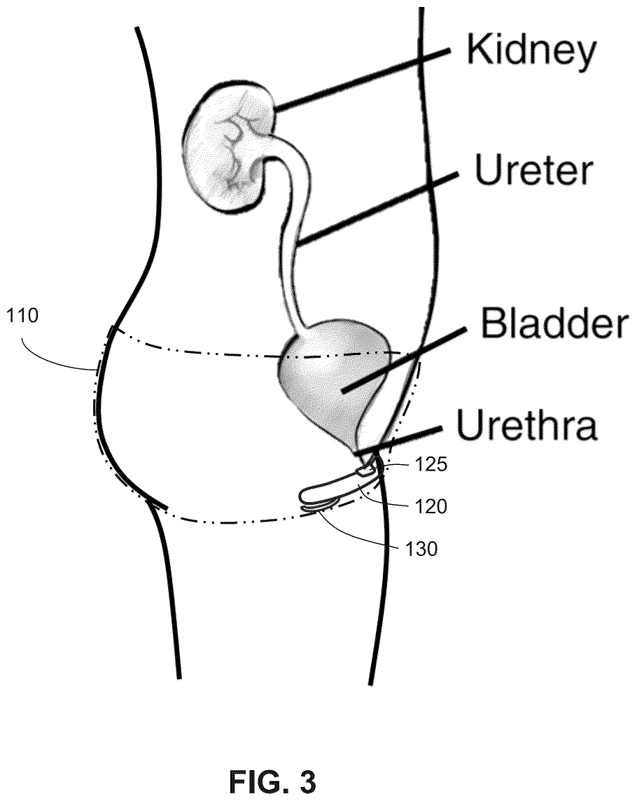

Referring to FIGS. 2-3, the present invention features a device that allows females to void their bladder while standing, sitting, walking, or any other body position or activity, eliminating the need for diapers, internal catheters, and artificial limitation of urine output. The devices provided herein can permit females to execute long flights, drives, walks, or any other activity that may restrict their ability to void while wearing clothes, flight suits, pressure suits, spacesuits, or others without relying on diapers. The devices also can keep females warmer in polar climates than they would be if they had to void in snow or blizzard-like conditions. The devices provided herein can be used in any situation where conventional urination methods are not possible or not ideal, such as for patients (e.g., Amyotrophic lateral sclerosis (ALS) patients) who are not bedridden but who have difficulty using a toilet or controlling urination.

In one aspect, this present invention features an external urinary device for use by a female. The device may comprise: (a) a restraint garment that contains a reservoir surrounded by a flexible lip; and (b) TWO or more hollow, open-ended tubes, wherein one tube comprises a proximal end connected to the reservoir and a lumen in fluid communication with the reservoir, wherein the article is adapted for reversible attachment of the lip to a female human body such that the reservoir is positioned over the urethral opening. The second tube proximal end is connected to the reservoir and a second lumen in fluid communication with the reservoir is to be connected to a one-way valve which allows air or other liquids/gasses the ability to balance/equalize any internal system pressures. The unit can be comprised of a soft flexible polymer (e.g., silicone).

In some embodiments, the lip surrounding the reservoir can be essentially any shape. The lip can be about five inches to about fifteen inches long and about 2 to about 6 inches wide. The lip can be about 0.05 to about 0.1 inches thick. The tube can be about one inch to about twenty inches long. The tubes can comprise a distal end, and wherein the device further comprises a fitting at the distal end and an extension tube, valve or other fitting can be attached to the distal fitting.

Although there has been shown and described the preferred embodiment of the present invention, it will be readily apparent to those skilled in the art that modifications may be made thereto which do not exceed the scope of the appended claims. Therefore, the scope of the invention is only to be limited by the following claims. In some embodiments, the figures presented in this patent application are drawn to scale, including the angles, ratios of dimensions, etc. In some embodiments, the figures are representative only and the claims are not limited by the dimensions of the figures. In some embodiments, descriptions of the inventions described herein using the phrase “comprising” includes embodiments that could be described as “consisting essentially of” or “consisting of”, and as such the written description requirement for claiming one or more embodiments of the present invention using the phrase “consisting essentially of” or “consisting of” is met.

The reference numbers recited in the below claims are solely for ease of examination of this patent application, and are exemplary, and are not intended in any way to limit the scope of the claims to the particular features having the corresponding reference numbers in the drawings.

What is claimed is:

1. A female urinary assistance system (100), the system (100) comprising a urine collection device comprising:
a. a primary reservoir (120) operatively coupled to the garment (110), configured to fluidly couple to a urethra of the user;
b. a flexible lip (125) disposed around a border of the primary reservoir (120), configured to create a waterproof seal between the user and the primary reservoir (120); and
c. a plurality of tubes (130) fluidly coupled to the primary reservoir (120) configured to siphon fluid from the primary reservoir (120).

2. The system (100) of claim 1, wherein the urine collection device further comprises a secondary reservoir (140), wherein at least one tube (132) of the plurality of tubes (130) is fluidly coupled to the secondary reservoir (140) such that the fluid is directed from the primary reservoir (120) to the secondary reservoir (140).

3. The system (100) of claim 1, wherein at least one tube (134) of the plurality of tubes (130) comprises a one-way valve (135) configured to direct an equalizing substance into the primary reservoir (120) such that an internal pressure of the primary reservoir (120) is balanced.

4. The system (100) of claim 3, wherein the equalizing substance comprises air, water, oxygen gas, or a combination thereof.

5. The system (100) of claim 1, wherein the flexible lip (125) comprises an adhering component configured to adhere the primary reservoir (120) to the user such that the waterproof seal is created.

6. The system (100) of claim 1, wherein the flexible lip (125) comprises silicon, natural rubber, engineered rubber, or a combination thereof.

7. The system (100) of claim 1, wherein the system (100) is configured to allow the user to urinate while sitting or standing in a vehicle, the vehicle comprising an aircraft, a spacecraft, an automobile, or a train.

8. The system (100) of claim 1, wherein at least one tube of the plurality of tubes (130) comprises a fitting (150) disposed at an end of the tube.

9. The system (100) of claim 8, wherein the fitting (150) comprises an extension tube, a valve, or a combination thereof.

10. The system (100) of claim 1, further comprising a restraint garment (110) for supporting the urine collection device against.

11. The system (100) of claim 10, wherein the restraint garment (110) comprises an absorbent material.

12. The system (100) of claim 10, wherein the restraint garment (110) is configured to fit around a pelvis of a user.

13. An external urinary assistance device (100) for permitting urination of a user having female urethral anatomy, the device (100) comprising:
a. a garment (110) configured to fit around a pelvis of the user;
b. a primary reservoir (120) operatively coupled to the garment (110), configured to fluidly couple to a urethra of the user;
c. a flexible lip (125) disposed around a border of the primary reservoir (120), the flexible lip (125) comprising an adhering component configured to adhere the primary reservoir (120) to the user such that the waterproof seal is created;
d. a plurality of tubes (130) fluidly coupled to the primary reservoir (120) configured to siphon fluid from the primary reservoir (120); and
e. a secondary reservoir (140), wherein at least one tube (132) of the plurality of tubes (130) are fluidly coupled to the secondary reservoir (140) such that the fluid is directed from the primary reservoir (120) to the secondary reservoir (140);
wherein at least one tube (134) of the plurality of tubes (130) comprise a one-way valve (135) configured to direct an equalizing substance into the primary reservoir (120) such that an internal pressure of the primary reservoir (120) is balanced.

14. The device (100) of claim 13, wherein the equalizing substance comprises air, water, oxygen gas, or a combination thereof.

15. The device (100) of claim 13, wherein the garment (110) comprises an absorbent material.

16. The device (100) of claim 13, wherein the flexible lip (125) comprises silicon, natural rubber, engineered rubber, or a combination thereof.

17. The device (100) of claim 13, wherein the device (100) is configured to allow the user to urinate while sitting or standing in a vehicle, the vehicle comprising an aircraft, a spacecraft, an automobile, or a train.

18. The device (100) of claim 13, wherein at least one tube of the plurality of tubes (130) comprises a fitting (150) disposed at an end of the tube.

19. The device (100) of claim 18, wherein the fitting (150) comprises an extension tube, a valve, or a combination thereof.

20. An external urinary assistance device (100) for permitting urination of a user having female urethral anatomy, the device (100) comprising:
a. an absorbent garment (110) configured to fit around a pelvis of the user;
b. a primary reservoir (120) operatively coupled to the absorbent garment (110), configured to fluidly couple to a urethra of the user;
c. a flexible silicon lip (125) disposed around a border of the primary reservoir (120), the flexible silicon lip (125) comprising an adhering component configured to adhere the primary reservoir (120) to the user such that the waterproof seal is created;
d. a plurality of tubes (130), each tube having a proximal end and a distal end, wherein the proximal end of each tube is fluidly coupled to the primary reservoir (120), wherein each tube is configured to siphon fluid from the primary reservoir (120); and
e. a secondary reservoir (140), wherein the distal end of at least one tube (132) of the plurality of tubes (130) is fluidly coupled to the secondary reservoir (140) such that the fluid is directed from the primary reservoir (120) to the secondary reservoir (140);
wherein at least one tube (134) of the plurality of tubes (130) comprises a one-way valve (135) configured to direct an equalizing substance into the primary reservoir (120) such that an internal pressure of the primary reservoir (120) is balanced;
wherein the device (100) is configured to allow the user to urinate while sitting or standing in a vehicle, the vehicle comprising an aircraft, a spacecraft, an automobile, or a train.
